(19) United States
(12) Patent Application Publication
(10) Pub. No.: US 2025/0288562 A1
(43) Pub. Date: Sep. 18, 2025

(54) Methods and Compositions for Treating Hidradenitis Suppurativa

(71) Applicant: Regranion, LLC, Mt. Pleasant, SC (US)

(72) Inventor: Gautam S. GHATNEKAR, Mt. Pleasant, SC (US)

(21) Appl. No.: 19/097,451

(22) Filed: Apr. 1, 2025

Related U.S. Application Data
(63) Continuation of application No. PCT/US23/34636, filed on Oct. 6, 2023.
(60) Provisional application No. 63/413,883, filed on Oct. 6, 2022.
(60) Provisional application No. 63/444,038, filed on Feb. 8, 2023.
(60) Provisional application No. 63/525,788, filed on Jul. 10, 2023.

(51) Int. Cl. A61K 31/437; A61P 17/00

(52) U.S. Cl. CPC A61K 31/437; A61P 17/00

(57) ABSTRACT

The present invention provides compositions and methods for treating hidradenitis suppurativa (HS) or an apocrine gland infection. In one embodiment, the invention provides a method for treating HS or an apocrine gland infection in a subject in need thereof, comprising administering to the subject a therapeutically effective amount of a compound of Formula (I),

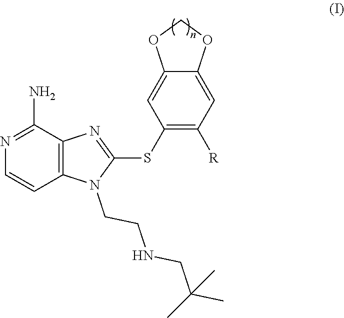

or a pharmaceutically acceptable salt thereof, wherein R is halogen, amino, —NH(C₁-C₃-alkyl) or —N(C₁-C₃-alkyl)₂ and n is 1 or 2.

### RELATED APPLICATIONS

This application is a continuation of International Application No. PCT/US23/34636, which designated the United States and was filed on Oct. 6, 2023, published in English, which claims the benefit of U.S. Provisional Application No. 63/413,883, filed on Oct. 6, 2022; U.S. Provisional Application No. 63/444,038, filed on Feb. 8, 2023; and U.S. Provisional Application No. 63/525,788, filed on Jul. 10, 2023. The entire teachings of the above applications are incorporated herein by reference.

### BACKGROUND OF THE INVENTION

Hidradenitis suppurativa (HS) is a chronic disease that affects 1-2% of the population with onset often occurring during childhood or adolescence. HS manifests as skin lesions including deep-seated nodules and abscesses, draining tracts and fibrotic scars, which occur most frequently in apocrine rich and intertriginous areas, such as axillary, groin, perianal, perineal, and inframammary locations. Treatment failure in response to adalimumab, the only FDA approved therapeutic for HS, is common (Saunte, D. M. L. & Jemec, G. B. E., J Am Med Assoc 318, 2019-2032 (2017)). Advances in understanding of HS pathophysiology have underscored resident and migrating cells of the innate immune system as well as IL-1b, TNFa, and the NRLP3 inflammasome as drivers of the inflammatory skin infiltration and destruction that underlies HS pathogenesis (Sabat, R. et al., Nature Reviews Disease Primers 6, 18 (2020)). While adalimumab and other drugs currently under development for HS target specific inflammatory cytokines, no therapies exist that uniformly downregulate the inflammatory mediators of HS, including IL-1b and the NRLP3 inflammasome (Saunte, D. M. L. & Jemec, G. B. E., ibid; Danby, F. W. & Margesson, L. J., Dermatol Clin 28, 779-793 (2010)). There is therefore a need for new compositions and methods for treating HS.

### SUMMARY OF THE INVENTION

The present invention provides compositions and methods for treating hidradenitis suppurativa (HS).

In one embodiment, the invention provides a method for treating HS in a subject in need thereof, comprising administering to the subject a therapeutically effective amount of a compound of Formula (I),

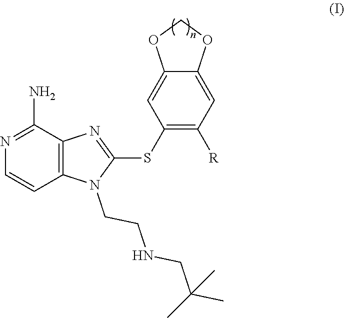

or a pharmaceutically acceptable salt thereof, wherein R is halogen, amino, —NH(C₁-C₃-alkyl) or —N(C₁-C₃-alkyl)₂ and n is 1 or 2.

In another embodiment, the invention provides a method of treating an apocrine gland infection in a nonhuman subject in need thereof, comprising administering to the subject a therapeutically effective amount of a compound of Formula (I) or a pharmaceutically acceptable salt thereof.

The invention further provides a pharmaceutical composition comprising a compound of Formula I, or a pharmaceutically acceptable salt thereof, for treating HS or an apocrine gland infection.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a graph showing the reduction of abscess/nodule (AN) count at week 16 relative to baseline for the Compound 1 and control groups in the study described in Example 1.

FIG. 2 is a graph showing the reduction of the HS physician's global assessment (HS-PGA) at week 16 relative to baseline for the Compound 1 and control groups in the study described in Example 1.

FIG. 3 is a graph showing the HS clinical response-50 (HiSCR-50) at week 16 relative to baseline for the Compound 1 and control groups in the study described in Example 1.

FIG. 4 is a graph showing the HS clinical response-75 (HiSCR-75) at week 16 relative to baseline for the Compound 1 and control groups in the study described in Example 1.

FIG. 5 is a graph showing the HS clinical response-90 (HiSCR-90) at week 16 relative to baseline for the Compound 1 and control groups in the study described in Example 1.

FIG. 6 is a graph showing the concentrations of Compound 1 found in the biopsied tissues in subjects grouped according to their clinical response to Compound 1 therapy.

### DETAILED DESCRIPTION OF THE INVENTION

The present invention relates to methods and compositions for treating HS. In one embodiment, the invention provides a method of treating HS in a human subject in need thereof, comprising administering to the subject a therapeutically effective amount of a compound of Formula I or a pharmaceutically acceptable salt thereof. The invention further relates to methods and compositions for treating an apocrine gland infection in a nonhuman subject.

Compounds of Formula I are potent inhibitors of heat shock protein 90 (HSP90), and the synthesis and HSP90 inhibitory activity of examples of certain compounds of Formula I are described in WO2008/115719, the contents of which are incorporated by reference herein in their entirety.

In preferred compounds of Formula (I), R is chloro, bromo, iodo, amino, methylamino or dimethylamino. In certain embodiments of the compound of Formula I, n is 1. In other embodiments of the compound of Formula I, n is 2. Suitable compounds of Formula I include, but are not limited to, the compounds set forth below.

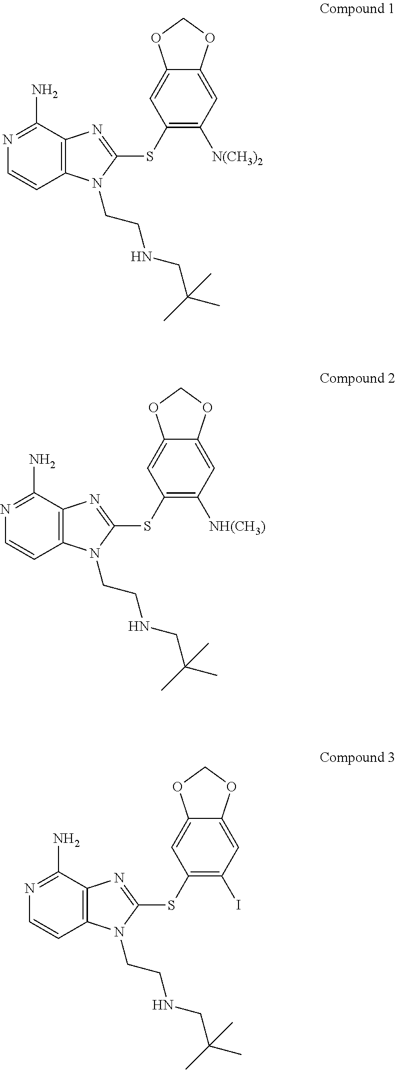

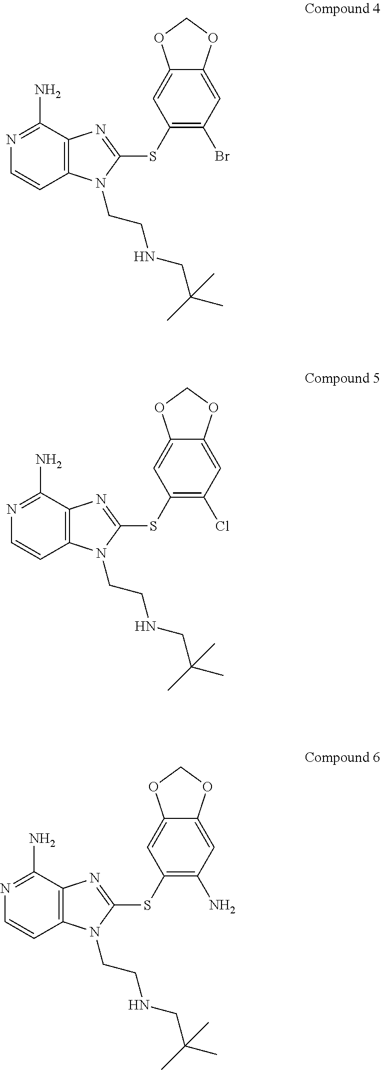 [SMILES: CC(C)(C)CNCCn1c(Sc2cc3c(cc2Br)OCO3)nc2c(N)nccc21 ; CC(C)(C)CNCCn1c(Sc2cc3c(cc2Cl)OCO3)nc2c(N)nccc21 ; CC(C)(C)CNCCn1c(Sc2cc3c(cc2N)OCO3)nc2c(N)nccc21]

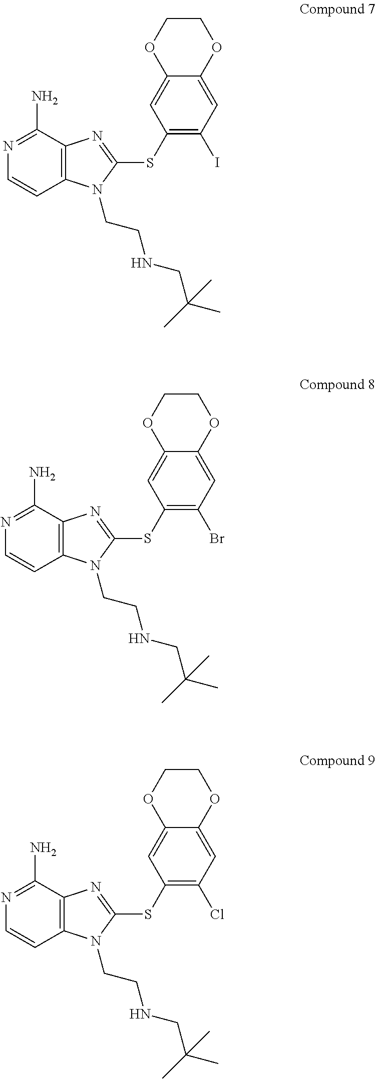 [SMILES: CC(C)(C)CNCCn1c(Sc2cc3c(cc2I)OCCO3)nc2c(N)nccc21 ; CC(C)(C)CNCCn1c(Sc2cc3c(cc2Br)OCCO3)nc2c(N)nccc21 ; CC(C)(C)CNCCn1c(Sc2cc3c(cc2Cl)OCCO3)nc2c(N)nccc21]

A particularly preferred compound of Formula I is Compound 1, which is also known as CUDC-305, Debio-0932, and RGRN-305.

In the methods of treating HS of the invention, the subject is preferably a human subject. The subject can have HS with symptoms at any degree of severity. For example, the subject can have Hurley stage I, II or III HS.

In the methods of treating an apocrine gland infection of the invention, the nonhuman subject is preferably a mammal, preferably a domesticated mammal. In certain embodiments, the subject is a companion or working animal, such as a domesticated canine (dog) or feline (cat), or a livestock animal, such as a bovine, ovine, equine, or porcine animal.

The compound of Formula I or pharmaceutically acceptable salt thereof is preferably administered in the form of a pharmaceutical composition comprising the therapeutic agent and a pharmaceutically acceptable carrier, excipient or diluent. Suitable pharmaceutical compositions include a solid, semisolid or liquid preparation (tablet, pellet, troche, capsule, suppository, cream, ointment, aerosol, powder, liquid, emulsion, suspension, syrup, injection, etc.). The pharmaceutical composition can be administered by any suitable means, including, without limitation, parenteral, intravenous, intramuscular, subcutaneous, implantation, oral, sublingual, buccal, nasal, pulmonary, transdermal, topical, vaginal, rectal, and transmucosal administrations or the like. Topical administration can involve the use of transdermal administration such as transdermal patches or iontophoresis devices.

In a preferred embodiment, the pharmaceutical composition is administered orally, for example, as a solid or a liquid preparation. Suitable solid oral formulations include tablets, capsules, pills, granules, pellets, sachets and effervescent, powders, and the like. Suitable liquid oral formulations include solutions, suspensions, dispersions, emulsions, oils and the like. In one embodiment of the present invention, the composition is formulated in a capsule. In another embodiment, the composition is formulated as a tablet.

Any inert excipient that is commonly used as a carrier or diluent may be used in the pharmaceutical compositions of the present invention, such as for example, a gum, a starch, a sugar, a cellulosic material, an acrylate, or mixtures thereof. A preferred diluent is microcrystalline cellulose. The compositions may further comprise a disintegrating agent (e.g., croscarmellose sodium) and a lubricant (e.g., magnesium stearate), and may additionally comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, polyethylene glycol), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In some embodiments, the active compound is prepared with a carrier that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art, for example, as described in U.S. Pat. No. 4,522,811.

It is especially advantageous to formulate oral compositions in unit dosage form for ease of administration and uniformity of dosage. “Unit dosage form”, as used herein, refers to physically discrete units suited as unitary dosages for the subject to be treated; each unit containing a predetermined quantity of active compound calculated to produce the desired therapeutic effect in association with the required pharmaceutical carrier. The specifications for the unit dosage forms of the invention are dictated by and directly dependent on the unique characteristics of the active compound and the particular therapeutic effect to be achieved, and the limitations inherent in the art of compounding such an active compound for the treatment of individuals.

Formulations of the invention intended for oral administration can include one or more permeation enhancers, including long chain fatty acids or salts thereof, such as decanoic acid and sodium decanoate.

The pharmaceutical compositions can be included in a container, pack, or dispenser together with instructions for administration.

The pharmaceutical composition of the invention is preferably suitable for oral administration and is most preferably in the form of a tablet. In one embodiment, the pharmaceutical composition is a tablet comprising Compound 1. In certain embodiments the tablet comprises Compound 1 in the form of the free base. In certain embodiments, the tablet comprises Compound 1 free base in an amount from 100 mg to 500 mg, from 150 mg to 450 mg, from 200 mg to 350 mg, from 200 mg to 300 mg, from 225 mg to 275 mg, 240 to 260 mg or 250 mg.

In certain embodiments, the tablet comprises Compound 1 free base in an amount from 0.5 mg to 300 mg, 0.5 mg to 250 mg, 0.5 mg to 225 mg, 0.5 mg to 200 mg, 0.5 mg to 175 mg, 0.5 mg to 150 mg, 0.5 mg to 125 mg, 0.5 mg to 100 mg, 0.5 mg to 75 mg, 0.5 mg to 50 mg, 0.5 mg to 20 mg, 0.5 mg to 15 mg, 0.5 mg to 10 mg, or 0.5 mg to 5 mg.

In certain embodiments, the tablet comprises Compound 1 free base in an amount from 1 mg to 300 mg, 1 mg to 250 mg, 1 mg to 225 mg, 1 mg to 200 mg, 1 mg to 175 mg, 1 mg to 150 mg, 1 mg to 125 mg, 1 mg to 100 mg, 1 mg to 75 mg, 1 mg to 50 mg, 1 mg to 20 mg, 1 mg to 15 mg, 1 mg to 10 mg, or 1 mg to 5 mg.

In certain embodiments, the tablet comprises Compound 1 free base in an amount in the amount of 10 mg to 400 mg. In certain embodiments, the tablet comprises Compound 1 free base in an amount from 10 mg to 300 mg, 10 mg to 250 mg, 10 mg to 225 mg, 10 mg to 200 mg, 10 mg to 175 mg, 10 mg to 150 mg, 10 mg to 125 mg, 10 mg to 100 mg, 10 mg to 75 mg, 10 mg in the amount of 50 mg, 10 mg to 20 mg, 10 mg to 15 mg per dosing day, or 10 mg.

In certain embodiments, the tablet comprises Compound 1 free base in the amount of 20 mg to 300 mg, 25 mg to 275 mg, 30 mg to 260 mg or 250 mg. In certain embodiments, the tablet comprises Compound 1 i 20 mg to 300 mg, 20 mg to 250 mg, 20 mg to 200 mg, 20 mg to 175 mg, 20 mg to 150 mg, 20 mg to 125 mg, 20 mg to 100 mg, 20 mg to 50 mg, 20 mg to 30 mg per dosing day, 25 mg per dosing day or 20 mg per dosing day. In certain embodiments, the tablet comprises Compound 1 in the amount of 50 mg to 150 mg, 60 mg to 140 mg, 70 mg to 130 mg, 75 mg to 125 mg, 80 mg to 120 mg, 90 mg to 110 mg or 100 mg. In certain embodiments, the tablet comprises Compound 1 in the amount of 50 mg to 200 mg, 60 mg to 190 mg, 70 mg to 180 mg, 80 mg to 170 mg, 90 mg to 160 mg, 100 mg to 150 mg, 110 mg to 140 mg, 115 mg to 135 mg or 125 mg.

Alternatively, the tablet comprises a pharmaceutically acceptable salt of Compound 1 in the foregoing amounts where the amounts represent free base equivalents. In certain embodiments, for example, the tablet comprises a free base equivalent amount of the pharmaceutically acceptable salt of Compound 1 from 0.5 mg to 125 mg or 0.5 to 250 mg.

The pharmaceutical composition can be administered daily or on a suitable schedule. In one embodiment, daily administration is repeated continuously for a period of several days to several years. Oral treatment may continue for between one week and the life of the patient. The administration can be continuous or intermittent, e.g., treatment for a number of consecutive days followed by a rest period.

The amount of the compound administered to the patient is preferably less than an amount that would cause toxicity in the patient. In certain embodiments, the amount of the compound of Formula I or pharmaceutically acceptable salt thereof that is administered to the patient is less than the amount that causes a concentration of the compound in the patient's plasma to equal or exceed the toxic level of the compound.

The dosing schedules described herein describe administration of Compound 1, which can be administered in the form of the free base or as a pharmaceutically acceptable salt. Any amount of Compound 1 administered in the dosing regimens described herein refers to the amount of the free base form of Compound 1, or with respect to a pharmaceutically acceptable salt, the free base equivalent amount.

In certain embodiments, Compound 1 is administered in the methods of the invention to a human subject at a dose of 0.5 mg to 400 mg per dosing day. Compound 1 is administered in an amount from 0.5 mg to 300 mg per dosing day, 0.5 mg to 250 mg per dosing day, 0.5 mg to 225 mg per dosing day, 0.5 mg to 200 mg per dosing day, 0.5 mg to 175 mg per dosing day, 0.5 mg to 150 mg per dosing day, 0.5 mg to 125 mg per dosing day, 0.5 mg to 100 mg per dosing day, 0.5 mg to 75 mg per dosing day, 0.5 mg to 50 mg per dosing day, 0.5 mg to 20 mg per dosing day, 0.5 mg to 15 mg per dosing day, 0.5 mg to 10 mg per dosing day, 0.5 mg to 5 mg per dosing day, or 0.5 to 2 mg per dosing day.

In certain embodiments, Compound 1 is administered in an amount from 1 mg to 300 mg per dosing day, 1 mg to 250 mg per dosing day, 1 mg to 225 mg per dosing day, 1 mg to 200 mg per dosing day, 1 mg to 175 mg per dosing day, 1 mg to 150 mg per dosing day, 1 mg to 125 mg per dosing day, 1 mg to 100 mg per dosing day, 1 mg to 75 mg per dosing day, 1 mg to 50 mg per dosing day, 1 mg to 20 mg per dosing day, 1 mg to 15 mg per dosing day, 1 mg to 10 mg per dosing day, or 1 mg to 5 mg per dosing day, or 1 mg per dosing day.

In certain embodiments, Compound 1 is administered at a dose of 10 mg to 400 mg per dosing day. In certain embodiments, Compound 1 is administered in an amount from 10 mg to 300 mg per dosing day, 10 mg to 250 mg per dosing day, 10 mg to 225 mg per dosing day, 10 mg to 200 mg per dosing day, 10 mg to 175 mg per dosing day, 10 mg to 150 mg per dosing day, 10 mg to 125 mg per dosing day, 10 mg to 100 mg per dosing day, 10 mg to 75 mg per dosing day, 10 mg to 50 mg per dosing day, 10 mg to 20 mg per dosing day, 10 mg to 15 mg per dosing day, or 10 mg per dosing day. In certain embodiments, Compound 1 is administered orally to the subject at a dose of 20 mg to 300 mg per dosing day, 25 mg to 275 mg per dosing day, 30 mg to 260 mg per dosing day or 250 mg per dosing day. In certain embodiments, Compound 1 is administered orally to the subject at a dose of 20 mg to 300 mg per dosing day, 20 mg to 250 mg per dosing day, 20 mg to 200 mg per dosing day, 20 mg to 175 mg per day, 20 mg to 150 mg per dosing day, 20 mg to 125 mg per dosing day, 20 mg to 100 mg per dosing day, 20 mg to 50 mg per dosing day, 20 mg to 30 mg per dosing day, 25 mg per dosing day or 20 mg per dosing day. In certain embodiments, Compound 1 is administered orally to the subject at a dose of 50 mg to 150 mg per dosing day, 60 mg to 140 mg per dosing day, 70 mg to 130 mg per dosing day, 75 mg to 125 mg per dosing day, 80 mg to 120 mg per dosing day, 90 mg to 110 mg per dosing day or 100 mg per dosing day. In certain embodiments, Compound 1 is administered orally to the subject at a dose of 50 mg to 200 mg per dosing day, 60 mg to 190 mg per dosing day, 70 mg to 180 mg per dosing day, 80 mg to 170 mg per dosing day, 90 mg to 160 mg per dosing day, 100 mg to 150 mg per dosing day, 110 mg to 140 mg per dosing day, 115 mg to 135 mg per dosing day or 125 mg per dosing day.

In certain embodiments, Compound 1 is administered orally to the human subject at a dose of 150 mg to 300 mg per dosing day, 200 mg to 300 mg per dosing day, 225 mg to 275 mg per dosing day, 240 mg to 260 mg per day or 250 mg per dosing day. The term “dosing day”, as used herein, refers to a day on which Compound 1, in the form of the free base or a pharmaceutically acceptable salt, is administered to the subject. In certain embodiments, Compound 1 is administered to the subject daily. In other embodiments, Compound 1 is administered every other day. In certain embodiments, Compound 1 is administered to the subject three days per week, preferably on non-consecutive days, for example on Monday, Wednesday, and Friday.

In certain embodiments, Compound 1 is initially administered to the human subject at a loading dose of 200-300 mg per dosing day for a suitable period of time, such as 1-12 weeks, 2-10 weeks or 4-8 weeks. The loading dose is preferably administered daily. The loading dose can be followed by a reduced maintenance dose, for example any of the reduced dosing schedules and/or reduced doses per dosing day as described above. For example, Compound 1 can be administered at loading dose of 200-250 mg per dosing day for 4 to 8 weeks, followed by a maintenance dose of 0.5-200 mg per dosing day, provided that the maintenance dose is a lower dose per dosing day and/or is dosed less frequently than the loading dose. In certain embodiments, the loading dose is 200 to 250 mg per day for 4 to 8 weeks and the maintenance dose is 0.5 mg to 200 mg or 20 mg to 200 mg per dosing day, provided that the maintenance dose is lower than the loading dose and/or is dosed less frequently that the loading dose.

Dosing of Compound 1 or a pharmaceutically acceptable salt thereof as described above can continue for any period of time and is preferably continued for at least 16 weeks or more. Preferably dosing continues as long as the subject is exhibiting clinical improvement in the signs and symptoms of HS.

Dosing of Compound 1 or a pharmaceutically acceptable salt thereof as described above can be interrupted by occasional periods of one or more days in which dosing is suspended, and then dosing can be continued either on a predetermined scheduled or upon return or worsening of HS signs and symptoms.

Response of a subject to the therapeutic methods of the present invention for treating HS can be determined as is known in the art. For example, the subject can be evaluated according to the Physician's Global Assessment (HS-PGA), Dermatology Life Quality Index (DLQI), and/or HS Clinical Response Score (HiSCR₅₀, HiSCR₇₅, HiSCR₉₀). (see Ann Intern Med,. 2012; 157:846-855; J Eur Acad Dermatol Venereol 201; 30:989-994; J Eur Acad Dermatol Venereol 2019; 33:1633-1643).

### Definitions

Listed below are definitions of various terms used to describe this invention. These definitions apply to the terms as they are used throughout this specification and claims, unless otherwise limited in specific instances, either individually or as part of a larger group.

The compound of Formula I can be formulated and administered in the methods of the invention as the free base or as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

The term “subject” as used herein refers to an animal. Preferably the animal is a mammal. Most preferably, the subject is a human. In certain embodiments, the subject is a companion or working animal, such as a domesticated canine (dog) or feline (cat), or a livestock animal, such as an equine, bovine, ovine or porcine animal.

The terms “therapeutically effective amount” and “effective amount” of a therapeutic agent refer to an amount of such agent which confers a therapeutic effect on the treated subject, at a reasonable benefit/risk ratio applicable to treatment of HS or an aprocrine gland infection. A therapeutically effective amount of an agent may be different when used as a single agent than when used in combination with one or more other agents. In addition, a therapeutically effective amount of an agent may depend on the specific combination of agents to be administered. The therapeutic effect may be objective (i.e., measurable by some test or marker) or subjective (i.e., subject gives an indication of or feels an effect). Therapeutically effective doses will also vary depending on route of administration, as well as the possibility of co-usage with other agents. It will be understood, however, that the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

Upon improvement of a patient's condition, a maintenance dose of a compound or composition of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Preparation of Compounds of Formula (I)

Compounds of Formula I, including compounds 1-9, can be prepared using the methods disclosed in published PCT Application No. WO2008/115719.

### EXAMPLES

### Example 1 Clinical Study of Compound 1 in Hidradenitis Suppurativa Patients

A placebo controlled clinical study was conducted to assess the efficacy of Compound 1 in HS patients. The study design is summarized below.

Fifteen patients were entered into the study and randomly assigned to the Compound 1 arm (10 patients) or the placebo arm (5 patients). The study was open to male or female subjects aged 18 years or older with hidradenitis suppurativa with minimum 5 inflammatory nodules in at least 2 distinct anatomic areas. The study was conducted in Denmark.

The study duration was approximately 24 weeks, including a 4-week screening phase, a 16-week treatment phase, and a 4-week observational follow-up phase to assess for hidradenitis suppurativa relapse and flare.

The primary objective of the study was to evaluate the efficacy of Compound 1 in subjects with moderate to severe hidradenitis suppurativa. The secondary objectives were to (1) evaluate the efficacy of Compound 1 on other clinical disease activity parameters in subject with moderate to severe hidradenitis suppurativa and (2) to evaluate the safety of Compound 1 in subjects with moderate to severe hidradenitis suppurativa.

The primary efficacy endpoint of the study was the percentage of subjects achieving Hidradenitis Suppurativa Clinical Response 50 (HiSCR-50) score at Week 16. The secondary efficacy endpoints were:
(1) the percentage of subjects with Hidradenitis Suppurativa Physician's Global Assessment (HS-PGA) scores of clear or minimal at Weeks 2, 4, 8, 12, 14, 16, and 20;
(2) the percentage of subjects achieving HiSCR-50, HiSCR-75, and HISCR-90 at Weeks 2, 4, 8, 12, 14, 16, and 20;
(3) changes from baseline in the Dermatology Life Quality Index (DLQI) total score at Weeks 4, 8, 12, 16, and 20;
(4) changes from baseline in the Pain Numerical Ranging score at Weeks 4, 8, 12, 16, and 20;
(5) change and percent change from Baseline in lesion counts (abscess count, inflammatory nodule count, AN count, and draining tunnel count);
(6) change from baseline in skin biomarkers at Week 16;
(7) change from baseline in blood biomarkers at Weeks 4, 8, 12, and 16.

The secondary safety endpoints were incidence of treatment-emergent adverse events (TEAEs) and incidence of related TEAEs.

### Overall Study Design

This was a 16-week treatment, randomized, double-blind, proof-of-concept study designed to assess the safety and efficacy of Compound 1 compared to placebo for use in future efficacy Phase 2 trials.

Eligible subjects completed an up-to 4-week screening phase and sequentially be randomized 2:1 to one of two cohorts, treatment cohort (n=10) or placebo-control cohort (n=5), in a 16-week treatment phase followed by a 4-week observational follow-up phase. In the 16-week treatment phase, subjects randomized to the treatment group receive 250 mg Compound 1 free base in the form of a tablet administered orally once daily. The 250 mg dose was selected based on the results of an earlier oncology trial conducted with doses in excess of 1000 mg/day as a safe minimal dose from which to conduct dose escalation if required. Subjects randomized to the placebo-control group receive placebo treatment once daily (microcrystalline cellulose, mannitol, crospovidone, Opadry II red, magnesium stearate, colloidal silicon dioxide).

HiSCR, HS-PGA, Pain-NRS, and DLQI were performed to assess efficacy. Safety was assessed with the vital signs, physical examination, clinical laboratory tests, ECG, and collection of AEs.

### Results

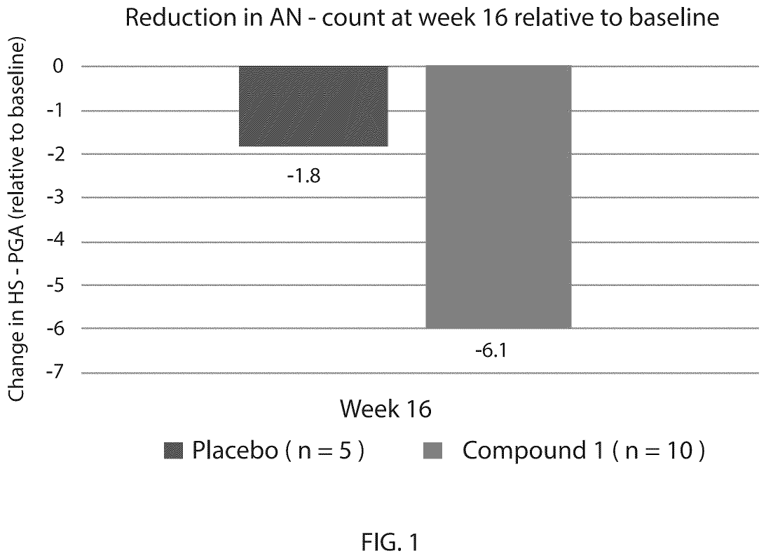

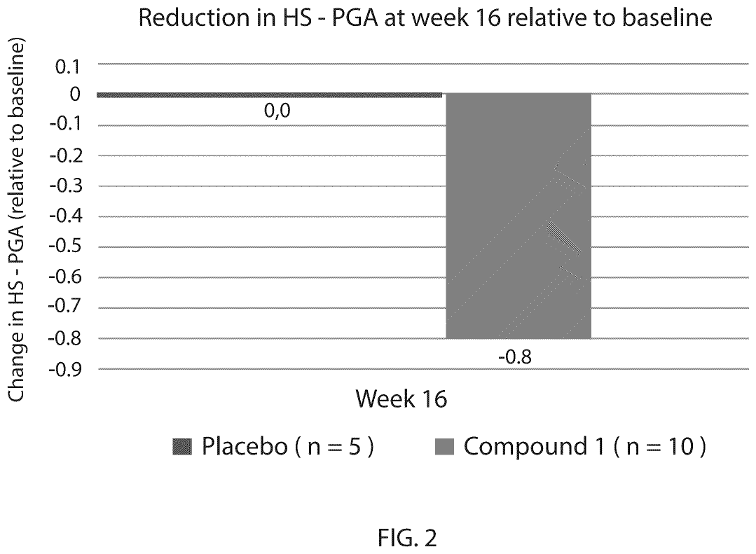

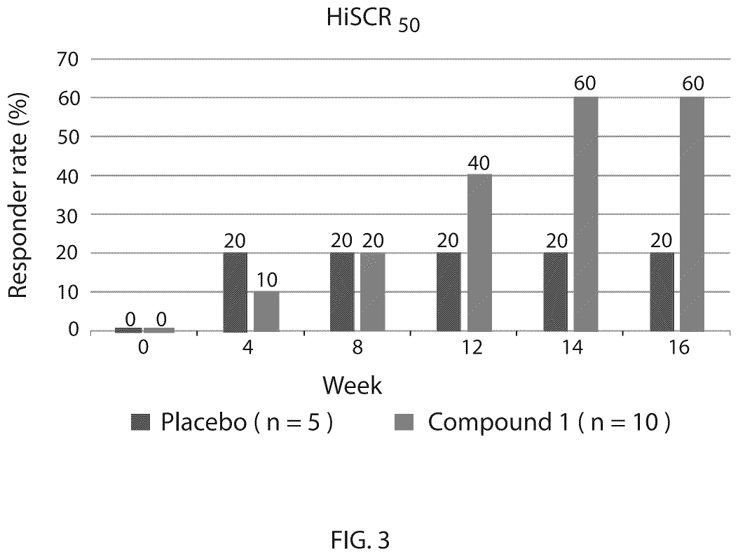

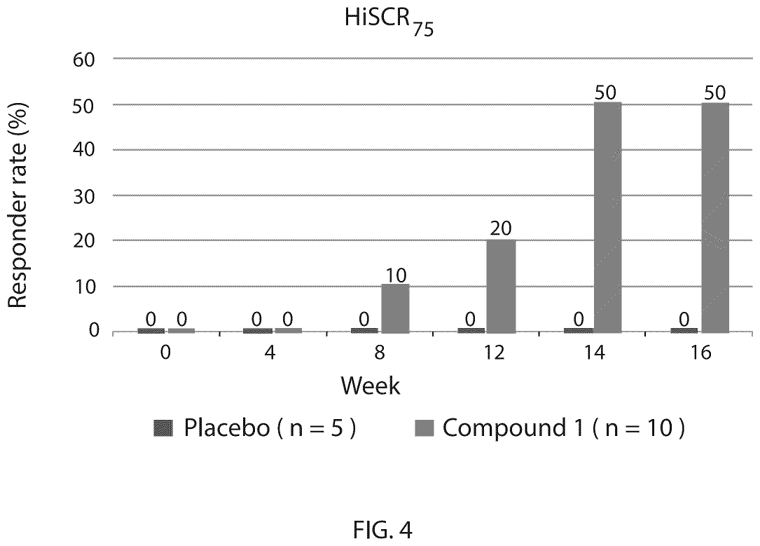

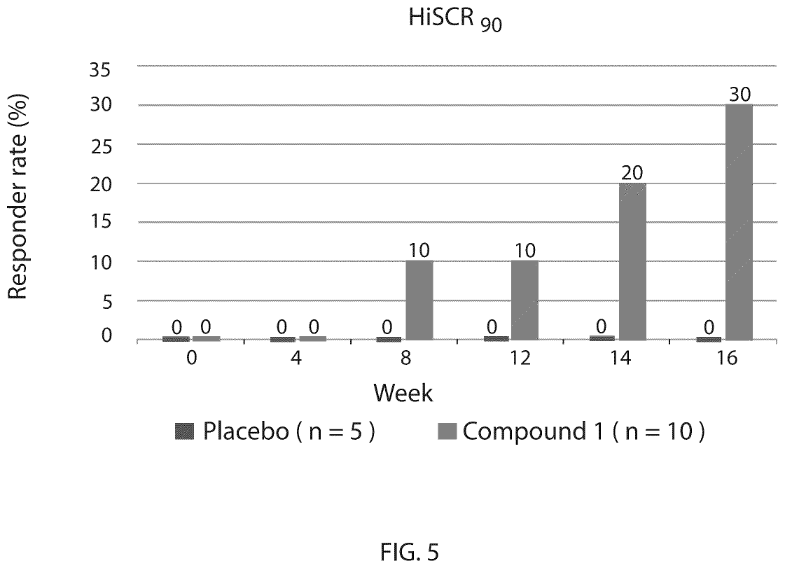

The results of the study are summarized in FIGS. 1-5. FIG. 1 shows that patients in the Compound 1 arm exhibited a substantially greater decrease in AN count relative to baseline at week sixteen compared to control. FIG. 2 shows the change in HS-PGA for patients in the Compound 1 and control arms. While the active arm showed an average decrease in HS-PGA of 0.8 compared to baseline, the control arm showed no change. FIGS. 3-5 respectively compare the HiSCR₅₀, HiSCR₇₅ and HiSCR₉₀ for subjects in the Compound 1 and control arms. Beginning at 12 weeks and continuing to 16 weeks, subjects in the Compound 1 arm were substantially more likely to achieve HiSCR₅₀. By 14 weeks, 50% of the subjects in the Compound 1 arm achieved HiSCR₇₅ compared to no subjects in the 5 control. Similarly, 30% of the subjects in the Compound 1 arm achieved HiSCR₉₀ by 16 weeks compared to no subjects in the control.

The table below shows the change from baseline in each subject's International Hidradentitis Suppurativa Severity Score System (IHS4) score at week 16. (See Zouboulis et al, Br J Dermatol 2017;177:1401-1409). Nine of the ten subjects in the Compound 1 arm showed a decrease in IHS4 score. In contrast, two of the subjects in the control arm showed modest decreases in IHS4 score, while one IHS4 score remained unchanged and two increased.

| Subject | IHS4 at baseline | IHS4 at week 16 |
|---|---|---|
| Compound 1 arm | | |
| 1 | 10 | 7 |
| 2 | 6 | 7 |
| 3 | 20 | 21 |
| 4 | 28 | 6 |
| 5 | 20 | 4 |
| 6 | 11 | 10 |
| 7 | 14 | 2 |
| 8 | 17 | 4 |
| 9 | 43 | 22 |
| 10 | 14 | 3 |
| Control arm | | |
| 1 | 25 | 25 |
| 2 | 10 | 6 |
| 3 | 30 | 36 |
| 4 | 8 | 7 |
| 5 | 10 | 16 |

### Example 2—Pharmacokinetic Studies of Compound 1

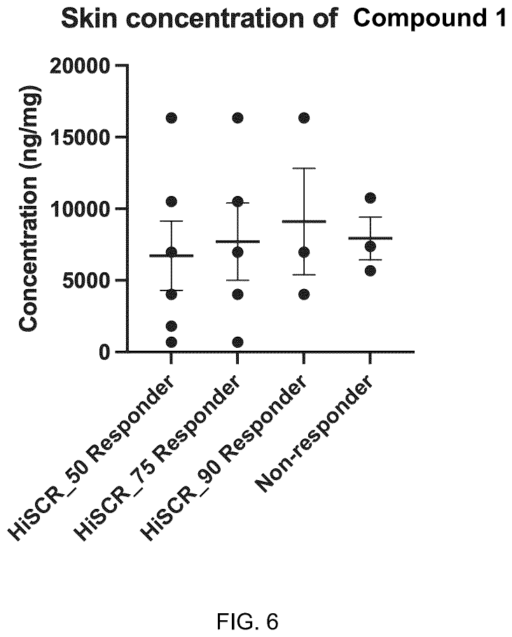

The accumulation of Compound 1 in the skin of human HS patients in the clinical study described in Example 1 was determined. Snap frozen skin punch biopsies obtained at the week 16 visit showed high concentrations of Compound 1, indicating that Compound 1 accumulates in the target tissue for HS therapy. FIG. 6 is a graph showing the concentrations of Compound 1 found in the biopsied tissues in subjects grouped according to their clinical response to Compound 1 therapy. The mean skin concentration following 16 weeks of treatment with Compound 1 was 7130 ng/mg, which is equivalent to 16.1 mM.

The skin accumulation found in this study was compared to plasma levels of Compound 1 determined in a previous study in healthy human volunteers. Six healthy male subjects were given a single oral dose of 250 mg Compound 1, and plasma samples were collected pre-dose and at 0.25, 0.5, 0.75, 1, 1.5, 2, 2.5, 3, 4, 6, 8, 10, 12, 18, 24, 30, 36, 48, 72 and 96 hours after dosing. The results of this study are shown in the table below.

| | AUCₗₐₛₜ | AUC_{∞} |
|---|---|---|
| Subject ID | (ng*hr/mL) | (ng*hr/mL) |
| 1 | 51.64 | 51.64 |
| 2 | 70.13 | 85.79 |
| 3 | 36.82 | 36.82 |
| 4 | 25.64 | 25.64 |
| 5 | 45.08 | 45.08 |
| 6 | 54.08 | 54.08 |

The Cₘₐₓ for Compound 1 determined for the six subjects ranged from 19.42 ng/ml to 24.92 ng/mL, with a median of 20.3 ng/ml or 46 nM.

The results show that the accumulation of Compound 1 is approximately 100,000-fold higher in the skin than in the plasma following daily oral dosing at 250 mg. In addition, the observed skin concentrations exceed the IC₅₀ of Compound 1 for inhibition of HSP-90 (0.1 μM) by a factor of about 10⁴. One of skill in the art would understand that a daily oral dose of Compound 1 which is significantly less than 250 mg, for example, as low as 1 mg per day, will result in sufficient skin accumulation of Compound 1 to inhibit HSP-90 in the skin and thereby treat HS.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts, and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for treating hidradenitis suppurativa in a subject in need thereof, comprising administering to the subject a therapeutically effective amount of a compound of Formula (I), 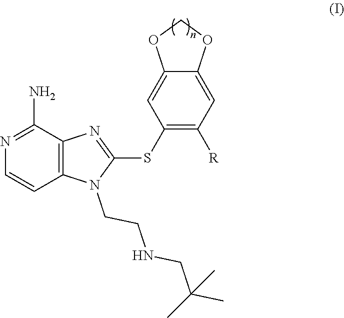
or a pharmaceutically acceptable salt thereof, wherein
R is halogen, amino, —NH(C₁-C₃-alkyl) or —N(C₁-C₃-alkyl)₂; and
n is 1 or 2.

2. (canceled)

3. The method of claim 1, wherein the compound of Formula (I) is

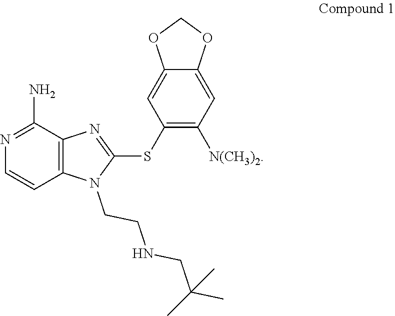 [SMILES: CN(C)c1cc2c(cc1Sc1nc3c(N)nccc3n1CCNCC(C)(C)C)OCO2]

4. The method of claim 1, wherein the subject has Hurley stage I hidradenitis suppurativa.

5. The method of claim 1, wherein the subject has Hurley stage II hidradenitis suppurativa or Hurley stage III hidradenitis suppurativa.

6. (canceled)

7. The method of claim 3, wherein Compound 1 or a pharmaceutically acceptable salt thereof is administered to the subject from 1 to 7 days per week.

8. The method of claim 7, wherein Compound 1 is administered orally to the subject as the free base at a dose of 0.5 mg to 400 mg per dosing day.

9. The method of claim 8, wherein Compound 1 is administered orally to the subject as the free base at a dose of 0.5 mg to 300 mg per dosing day, 0.5 mg to 250 mg per dosing day, 0.5 mg to 225 mg per dosing day, 0.5 mg to 200 mg per dosing day, 0.5 mg to 175 mg per dosing day, 0.5 mg to 150 mg per dosing day, 0.5 mg to 125 mg per dosing day, 0.5 mg to 100 mg per dosing day, 0.5 mg to 75 mg per dosing day, 0.5 mg to 50 mg per dosing day, 0.5 mg to 20 mg per dosing day, 0.5 mg to 15 mg per dosing day, 0.5 mg to 10 mg per dosing day, 0.5 mg to 5 mg per dosing day, 0.5 mg to 3 mg per dosing day, or 0.5 mg to 2 mg per dosing day.

10. The method of claim 8, wherein Compound 1 is administered orally to the subject as the free base at a dose of 1 mg to 300 mg per dosing day, 1 mg to 250 mg per dosing day, 1 mg to 225 mg per dosing day, 1 mg to 200 mg per dosing day, 1 mg to 175 mg per dosing day, 1 mg to 150 mg per dosing day, 1 mg to 125 mg per dosing day, 1 mg to 100 mg per dosing day, 1 mg to 75 mg per dosing day, 1 mg to 50 mg per dosing day, 1 mg to 20 mg per dosing day, 1 mg to 15 mg per dosing day, 1 mg to 10 mg per dosing day, or 1 mg to 5 mg per dosing day, or 1 mg per dosing day.

11. The method of claim 8, wherein Compound 1 is administered orally to the subject as the free base at a dose of 10 mg to 400 mg per dosing day, 10 mg to 300 mg per dosing day, 10 mg to 250 mg per dosing day, 10 mg to 225 mg per dosing day, 10 mg to 200 mg per dosing day, 10 mg to 175 mg per dosing day, 10 mg to 150 mg per dosing day, 10 mg to 125 mg per dosing day, 10 mg to 100 mg per dosing day, 10 mg to 75 mg per dosing day, 10 mg to 50 mg per dosing day, 10 mg to 20 mg per dosing day, 10 mg to 15 mg per dosing day, or 10 mg per dosing day.

12. The method of claim 8, wherein Compound 1 is administered orally to the subject at a dose of 225 mg to 275 mg per day, 240 mg to 260 mg per day, 245 to 255 mg/day or 250 mg per day.

13. The method of claim 7, wherein a pharmaceutically acceptable salt of Compound 1 is administered orally to the subject at a free base equivalent dose of 0.5 mg to 400 mg per dosing day.

14. The method of claim 13, wherein the pharmaceutically acceptable salt of Compound 1 is administered orally to the subject at a free base equivalent dose of 0.5 mg to 300 mg per dosing day, 0.5 mg to 250 mg per dosing day, 0.5 mg to 225 mg per dosing day, 0.5 mg to 200 mg per dosing day, 0.5 mg to 175 mg per dosing day, 0.5 mg to 150 mg per dosing day, 0.5 mg to 125 mg per dosing day, 0.5 mg to 100 mg per dosing day, 0.5 mg to 75 mg per dosing day, 0.5 mg to 50 mg per dosing day, 0.5 mg to 20 mg per dosing day, 0.5 mg to 15 mg per dosing day, 0.5 mg to 10 mg per dosing day, 0.5 mg to 5 mg per dosing day, 0.5 mg to 3 mg per dosing day, or 0.5 mg to 2 mg per dosing day.

15. The method of claim 13, wherein the pharmaceutically acceptable salt of Compound 1 is administered orally to the subject at a free base equivalent dose of 1 mg to 300 mg per dosing day, 1 mg to 250 mg per dosing day, 1 mg to 225 mg per dosing day, 1 mg to 200 mg per dosing day, 1 mg to 175 mg per dosing day, 1 mg to 150 mg per dosing day, 1 mg to 125 mg per dosing day, 1 mg to 100 mg per dosing day, 1 mg to 75 mg per dosing day, 1 mg to 50 mg per dosing day, 1 mg to 20 mg per dosing day, 1 mg to 15 mg per dosing day, 1 mg to 10 mg per dosing day, or 1 mg to 5 mg per dosing day, or 1 mg per dosing day.

16. The method of claim 13, wherein the pharmaceutically acceptable salt of Compound 1 is administered orally to the subject at a free base equivalent dose of 10 mg to 400 mg per dosing day, 10 mg to 300 mg per dosing day, 10 mg to 250 mg per dosing day, 10 mg to 225 mg per dosing day, 10 mg to 200 mg per dosing day, 10 mg to 175 mg per dosing day, 10 mg to 150 mg per dosing day, 10 mg to 125 mg per dosing day, 10 mg to 100 mg per dosing day, 10 mg to 75 mg per dosing day, 10 mg to 50 mg per dosing day, 10 mg to 20 mg per dosing day, 10 mg to 15 mg per dosing day, or 10 mg per dosing day.

17. The method of claim 13, wherein the pharmaceutically acceptable salt of Compound 1 is administered orally to the subject at a free base equivalent dose of 225 mg to 275 mg per day, 240 mg to 260 mg per day, 245 to 255 mg/day or 250 mg per day.

18. The method of claim 1, wherein the compound of Formula (I) or pharmaceutically acceptable salt thereof is orally administered in the form of a tablet.

19. The method of claim 18, wherein the compound of Formula (I) is Compound 1 and the tablet comprises 0.5 mg to 300 mg free base equivalent amount of Compound 1 or a pharmaceutically acceptable salt thereof.

20. The method of claim 13, wherein the free base equivalent amount of Compound 1 or pharmaceutically acceptable salt thereof is 1 to 275 mg, 10 to 150 mg, 50 to 150 mg, or 100 mg.

21. The method of claim 14, wherein the free base equivalent amount of Compound 1 or pharmaceutically acceptable salt thereof is 125 or 250 mg.

22. The method of claim 12, wherein the tablet comprises Compound 1 free base in the amount of 10, 50, 75, 100, 125, 150, 175, 200, 225 or 250 mg.

23. (canceled)
